SEQUENCE LISTING (1 sequence)

SEQ ID NO: 1 - DNA, 1668 bp, Bacillus subtilis
agctcggatccactagtaacggccgccagtgtgctggaattcgcccttagaaaggaggtgatccagccgcaccttccgatacggctaccttgttacgacttcaccccaatcatctgtcccaccttcggcggctggctccataaaggttacctcaccgacttcgggtgttacaaactctcgtggtgtgacgggcggtgtgtacaaggcccgggaacgtattcaccgcggcatgctgatccgcgattactagcgattccagcttcacgcagtcgagttgcagactgcgatccgaactgagaacagatttgtgrgattggcttaacctcgcggtttcgctgccctttgttctgtccattgtagcacgtgtgtagcccaggtcataaggggcatgatgatttgacgtcatccccaccttcctccggtttgtcaccggcagtcaccttagagtgcccaactgaatgctggcaactaagatcaagggttgcgctcgttgcgggacttaacccaacatctcacgacacgagctgacgacaaccatgcaccacctgtcactctgcccccgaaggggacgtcctatctctaggattgtcagaggatgtcaagacctggtaaggttcttcgcgttgcttcgaattaaaccacatgctccaccgcttgtgcgggcccccgtcaattcctttgagtttcagtcttgcgaccgtactccccaggcggagtgcttaatgcgttagctgcagcactaaaggggcggaaaccccctaacacttagcactcatcgtttacggcgtggactaccagggtatctaatcctgttcgctccccacgctttcgctcctcagcgtcagttacagaccagagagtcgccttcgccactggtgttcctccacatctctacgcatttcaccgctacacgtggaattccactctcctcttctgcactcaagttccccagtttccaatgaccctccccggttgagccgggggctttcacatcagacttaagaaaccgcctgcgagccctttacgcccaataattccggacaacgcttgccacctacgtattaccgcggctgctggcacgtagttagccgtggctttctggttaggtaccgtcaaggtgccgccctatttgaacggcacttgttcttccctaacaacagagctttacgatccgaaaaccttcatcactcacgcggcgttgctccgtcagactttcgtccattgcggaagattccctactgctgcctcccgtaggagtctgggccgtgtctcagtcccagtgtggccgatcaccctctcaggtcggctacgcatcgtcgccttggtgagccgttacctcaccaactagctaatgcgccgcgggtccatctgtaagtggtagccgaagccaccttttatgtctgaaccatgcggttcagacaaccatccggtattagccccggtttcccggagttatcccagtcttacaggcaggttacccacgtgttactcacccgtccgccgctaacatcagggagcaagctcccatctgtccgctcgacttgcatgtattaggcacgccgccagcgttcgtcctgagccatgaacaaactctaagggcgaattctgcagatatccatcacactggcggccgctcgagcatgcatctagagggcccaatcgccctat

(19) United States
(12) Patent Application Publication
(10) Pub. No.: US 2019/0231882 A1
(43) Pub. Date: Aug. 1, 2019

(54) METHODS FOR STABILIZING BACTERIAL PROBIOTIC COMPOSITIONS

(71) Applicant: BiOWiSH Technologies Inc., Cincinnati, OH (US)

(72) Inventors: Michael Stanford Showell, Cincinnati, OH (US); John Gorsuch, Cincinnati, OH (US); Melanie Jeffries, Cincinnati, OH (US)

(21) Appl. No.: 16/262,534

(22) Filed: Jan. 30, 2019

Related U.S. Application Data
(60) Provisional application No. 62/623,733, filed on Jan. 30, 2018.

(51) Int. Cl. A61K 47/36; A61K 35/742; A61K 9/00; A61P 1/00; A61P 1/12; A61K 35/744; A61K 35/747; A61K 45/06; A61P 1/14; A23L 33/135

(52) U.S. Cl. CPC A61K 47/36; A61K 35/742; A61K 9/0053; A61K 9/0056; A61K 9/0095; A61P 1/00; A61K 2035/115; A61K 35/744; A61K 35/747; A61K 45/06; A61P 1/14; A23L 33/135; A61P 1/12

(57) ABSTRACT

The present invention relates to stabilized probiotic compositions that include lactic-acid-producing bacteria. The present invention also relates to methods for stabilizing lactic-acid-producing bacteria with a diluent having a plurality of monosaccharide units connected predominantly by β-glycosidic bonds or α-glycosidic bonds that are resistant to hydrolysis by an amylase.

### CROSS-REFERENCE TO RELATED APPLICATIONS

This application claims the benefit of and priority to U.S. Provisional Application No. 62/623,733, filed on Jan. 30, 2018, the contents of which are hereby incorporated by reference in their entireties.

### INCORPORATION BY REFERENCE OF SEQUENCE LISTING

The contents of the text file named “BIOW-017 SEQ_LISTING.txt”, which was created on Dec. 18, 2018 and is 2.47 KB in size, are hereby incorporated by reference in their entireties.

### FIELD OF THE INVENTION

The present invention relates to methods for stabilizing lactic-acid-producing bacteria. The present invention also relates to stabilized probiotic compositions that include one or more lactic-acid-producing bacteria.

### BACKGROUND OF THE INVENTION

The use of probiotic formulations to improve human and/or animal health and nutrition is well known in the art. These formulations typically include lactic-acid-producing bacteria such as Lactobacillus Plantarum. Stability of these bacteria in human probiotic and animal feed compositions at the point of consumption is one of the major challenges associated with their use. The bacterial strains used in probiotic formulations tend to be fastidious, nutritionally demanding and sensitive to environmental and processing conditions.

Accordingly, there is a need for methods to stabilize lactic-acid-producing bacteria.

### SUMMARY OF THE INVENTION

One aspect of the invention relates to a dry composition including: (a) 5% or less by weight of a bacterial composition containing one or more lactic-acid-producing bacteria having a bacterial concentration of at least 1×10⁶ colony forming units (CFU) per gram of the bacterial composition; and (b) at least 95% by weight of a diluent, wherein the diluent comprises a plurality of monosaccharide units connected by a plurality of linkages, each monosaccharide unit being connected to an adjacent monosaccharide unit by one of the linkages, wherein at least 70% of the linkages are β-glycosidic bonds, and wherein at least 80% of the bacterial activity in the dry composition is retained over a period of 30 days.

In some embodiments, the bacterial composition further includes a Bacillus bacterial species such as Bacillus subtilis 34 KLB.

In some embodiments, at least 80% or at least 90% of the linkages are β-glycosidic bonds.

In some embodiments, the β-glycosidic bond is selected from the group consisting of a β-1,1 linkage, a β-1,2 linkage, a β-1,3 linkage, a β-1,4 linkage, a β-1,5 linkage, and a β-1,6 linkage.

In some embodiments, the one or more lactic-acid-producing bacteria is Lactobacillus Plantarum, Pediococcus acidilactici, Pediococcus pentosaceus, or a mixture thereof. Each of the Pediococcus acidilactici, Pediococcus pentosaceus, and Lactobacillus plantarum can be fermented anaerobically, dried, and ground to an average particle size of about 200 microns.

In some embodiments, the Lactobacillus Plantarum, Pediococcus acidilactici, Pediococcus pentosaceus are present at equal CFU count per gram of the composition.

In some embodiments, the diluent is carboxymethylcellulose, chitosan, chrysoloaminarin, curdlan, laminarin, lentinan, lichenin, pleuran, zymosan, oat beta glucan, wheat beta glucan, rye beta glucan, barley beta glucan, chondroitin sulfate, or a mixture thereof.

In some embodiments, the dry composition can further include (a) a prebiotic selected from the group consisting of inulin, a fructooliogsaccharide, and a glucooligosaccharide, and/or (b) a vitamin, a mineral, a sugar, a botanical, or a fungal component. The vitamin can be vitamin A, vitamin B1, vitamin B2, vitamin B3, vitamin B5, vitamin B6, vitamin B7, vitamin B9, vitamin B12, vitamin C, vitamin D, vitamin E, or vitamin K. The mineral can be diatomaceous earth, calcium carbonate, calcium lactate, calcium chloride, calcium phosphate (dibasic), sodium chloride, potassium citrate monohydrate, potassium sulfate, potassium phosphate monobasic, magnesium oxide, manganese carbonate, manganese gluconate, ferric citrate, zinc carbonate, zinc gluconate, cupric carbonate, potassium iodate, sodium selenite pentahydrate, chromium potassium sulfate dodecahydrate, ammonium paramolybdate tetrahydrate, sodium meta-silicate nonahydrate, lithium chloride, boric acid, sodium fluoride, nickel carbonate hydroxide tetrahydrate, or ammonium meta-vanadate. The botanical can be rhodiola rosea extract, acadia catechu, scutellaria baicalensis, moringa, or turmeric. The fungal component can be ganoderma lucidium, lentinus edodes, hericium erinaceuous, agaricus blazei, cordyceps sinensis, coriolus versicolor, corprinus comatus, or grifola frondosa.

In some embodiments, the dry composition has a moisture content of less than 5% by weight.

Another aspect of the invention relates to a dry composition including: (a) 5% or less by weight of a bacterial composition containing one or more lactic-acid-producing bacteria having a bacterial concentration of at least 1×10⁶ colony forming units (CFU) per gram of the bacterial composition; and (b) at least 95% by weight of a diluent, wherein the diluent includes a plurality of monosaccharide units connected by a plurality of linkages, each monosaccharide unit being connected to an adjacent monosaccharide unit by one of the linkages, where at least 70% of the linkages are α-glycosidic bonds that are resistant to hydrolysis by an amylase, and wherein at least 80% of the bacterial activity in the dry composition is retained over a period of 30 days.

In some embodiments, the bacterial composition further includes a Bacillus bacterial species such as Bacillus subtilis 34 KLB.

In some embodiments, at least 80% or at least 90% of the linkages are α-glycosidic bonds that are resistant to hydrolysis by an amylase.

In some embodiments, the α-glycosidic bond is selected from the group consisting of an α-1,1 linkage, an α-1,2 linkage, an α-1,3 linkage, an α-1,4 linkage, an α-1,5 linkage, and an α-1,6 linkage.

In some embodiments, the one or more lactic-acid-producing bacteria is Lactobacillus Plantarum, Pediococcus acidilactici, Pediococcus pentosaceus, or a mixture thereof. Each of the Pediococcus acidilactici, Pediococcus pentosaceus, and Lactobacillus plantarum can be fermented anaerobically, dried, and ground to an average particle size of about 200 microns.

In some embodiments, the Lactobacillus Plantarum, Pediococcus acidilactici, Pediococcus pentosaceus are present at equal CFU count per gram of the composition.

In some embodiments, the diluent is digestion-resistant maltodextrin, digestion-resistant inulin, digestion-resistant starch, or a mixture thereof.

In some embodiments, the dry composition can further include (a) a prebiotic selected from the group consisting of inulin, a fructooliogsaccharide, and a glucooligosaccharide, and/or (b) a vitamin, a mineral, a sugar, a botanical, or a fungal component. The vitamin can be vitamin A, vitamin B1, vitamin B2, vitamin B3, vitamin B5, vitamin B6, vitamin B7, vitamin B9, vitamin B12, vitamin C, vitamin D, vitamin E, or vitamin K. The mineral can be diatomaceous earth, calcium carbonate, calcium lactate, calcium chloride, calcium phosphate (dibasic), sodium chloride, potassium citrate monohydrate, potassium sulfate, potassium phosphate monobasic, magnesium oxide, manganese carbonate, manganese gluconate, ferric citrate, zinc carbonate, zinc gluconate, cupric carbonate, potassium iodate, sodium selenite pentahydrate, chromium potassium sulfate dodecahydrate, ammonium paramolybdate tetrahydrate, sodium meta-silicate nonahydrate, lithium chloride, boric acid, sodium fluoride, nickel carbonate hydroxide tetrahydrate, or ammonium meta-vanadate. The botanical can be rhodiola rosea extract, acadia catechu, scutellaria baicalensis, moringa, or turmeric. The fungal component can be ganoderma lucidium, lentinus edodes, hericium erinaceuous, agaricus blazei, cordyceps sinensis, coriolus versicolor, corprinus comatus, or grifola frondosa.

In some embodiments, the dry composition has a moisture content of less than 5% by weight.

The dry compositions disclosed herein can be used to (a) improve a subject's health or nutrition, (b) treat a digestive disorder in a subject in need thereof, and/or (c) feed an animal. In some embodiments, the dry composition is administered orally. In some embodiments, the dry composition is administered through animal feed or drinking water. The composition can be in the form of a tablet, capsule, powder, or granulate. Each tablet, capsule, powder, or granulate can contain between 100-800 milligrams of the composition. In some embodiments, the digestive disorder is constipation, diarrhea, dysbiosis, Crohn's disease, food allergy, lactose intolerance, pouchitis, or ulcerative colitis.

Another aspect of the invention relates to a method of stabilizing lactic-acid-producing bacteria, the method including contacting the lactic-acid-producing bacteria with a diluent having a plurality of monosaccharide units connected by a plurality of linkages, each monosaccharide unit being connected to an adjacent monosaccharide unit by one of the linkages, wherein at least 70% of the linkages are β-glycosidic bonds.

Yet another aspect of the invention relates to a method of stabilizing lactic-acid-producing bacteria, the method including contacting the lactic-acid-producing bacteria with a diluent having a plurality of monosaccharide units connected by a plurality of linkages, each monosaccharide unit being connected to an adjacent monosaccharide unit by one of the linkages, wherein at least 70% of the linkages are α-glycosidic bonds that are resistant to hydrolysis by an amylase.

In some embodiments, the methods disclosed herein can retain at least 80% or at least 90% of the bacterial activity.

### BRIEF DESCRIPTION OF THE DRAWINGS

FIG. 1 shows stability of several lactic-acid-producing bacteria formulations with different diluents after 30 days at 30° C. “CMC” stands for carboxymethylcellulose.

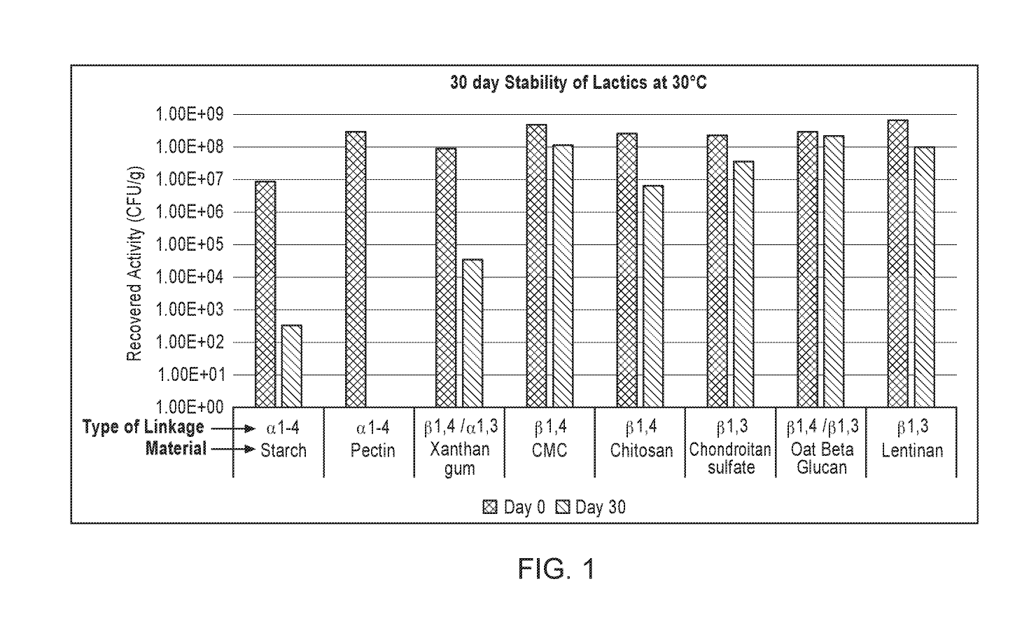

FIG. 2 shows the effect of increasing levels of dextrose on the stability of a blend of lactic-acid-producing bacteria.

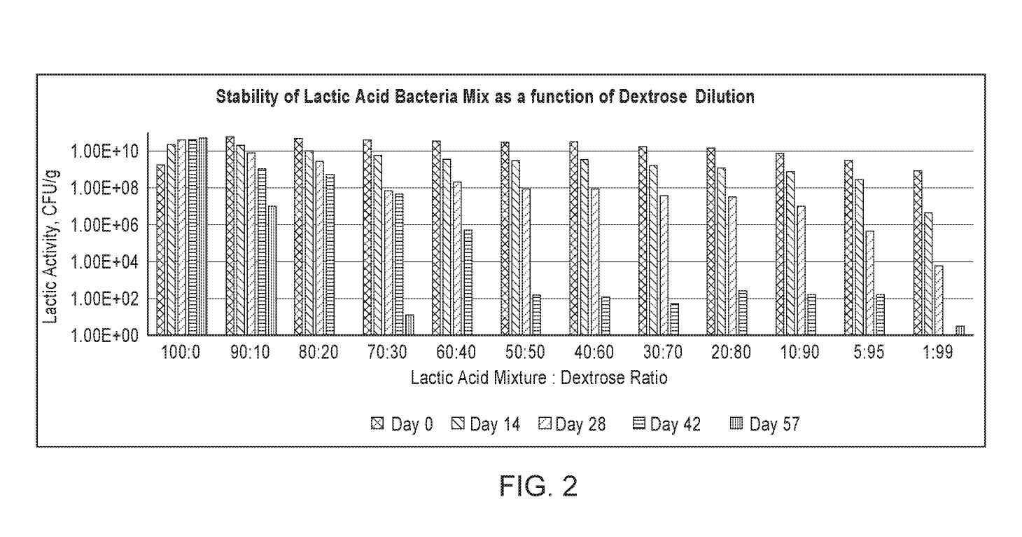

FIG. 3 shows stability out to 96 days at 30° C. for several lactic-acid-producing bacteria formulations.

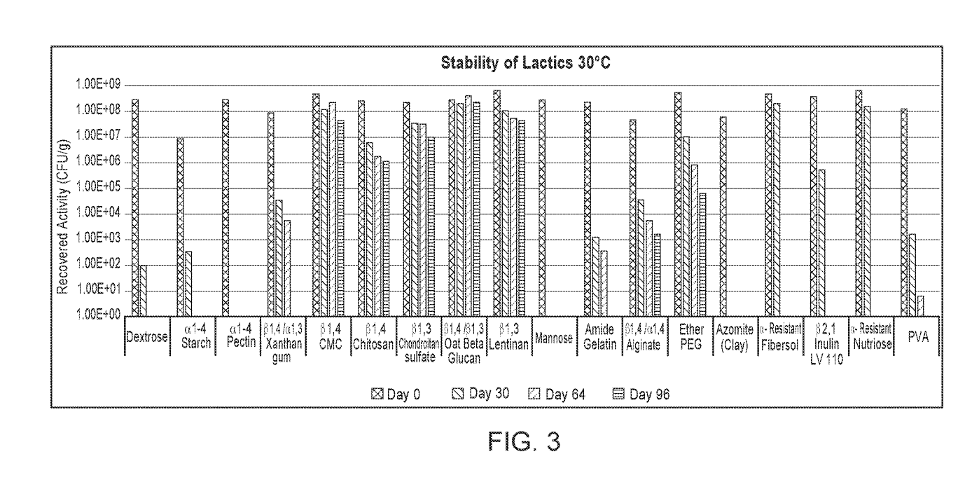

### DETAILED DESCRIPTION OF THE INVENTION

The present disclosure is based, inter alia, on the discovery that diluents rich in β-glycosidic bonds can stabilize lactic-acid-producing bacteria. Alternatively, it was discovered that diluents rich in α-glycosidic bonds that are resistant to hydrolysis by an amylase can also stabilize lactic-acid-producing bacteria. Accordingly, the present disclosure provides dry compositions that include lactic-acid-producing bacteria. In one aspect, the present disclosure provides a dry composition that includes: (a) a bacterial composition including one or more lactic-acid-producing bacteria having a bacterial concentration of at least 1×10⁶ colony forming units (CFU) per gram of the bacterial composition; and (b) at least 95% by weight of a diluent, wherein the diluent comprises a plurality of monosaccharide units connected by a plurality of linkages, each monosaccharide unit being connected to an adjacent monosaccharide unit by one of the linkages, wherein at least 70% of the linkages are β-glycosidic bonds. The dry composition is stable, e.g., at room temperature. For example, at least 80% of the bacterial activity is retained over a period of at least 15 days, e.g., at least 20 days, at least 25 days, at least 30 days, at least 35 days, at least 40 days, at least 45 days, at least 50 days, at least 55 days, or at least 60 days. In some embodiments, at least 90% of the bacterial activity is retained over a period of at least 15 days, e.g., at least 20 days, at least 25 days, at least 30 days, at least 35 days, at least 40 days, at least 45 days, at least 50 days, at least 55 days, or at least 60 days. In some embodiments, at least 95% of the bacterial activity is retained over a period of at least 15 days, e.g., at least 20 days, at least 25 days, at least 30 days, at least 35 days, at least 40 days, at least 45 days, at least 50 days, at least 55 days, or at least 60 days. The bacterial activity in the dry composition can be determined by the bacterial concentration. For example, the bacterial activity or bacterial concentration can be measured by traditional plate counting using agar, such as De Man, Rogosa and Sharpe (MRS) agar.

Non-limiting examples of monosaccharide units include glucose, fructose, and galactose. The diluent can include the same type of monosaccharide unit or two or more different types of monosaccharide units.

In some embodiments, at least 75% of the linkages are β-glycosidic bonds. In some embodiments, at least 80% of the linkages are β-glycosidic bonds. In some embodiments, at least 85% of the linkages are β-glycosidic bonds. In some embodiments, at least 90% of the linkages are β-glycosidic bonds. In some embodiments, at least 91% of the linkages are β-glycosidic bonds. In some embodiments, at least 92% of the linkages are β-glycosidic bonds. In some embodiments, at least 93% of the linkages are β-glycosidic bonds. In some embodiments, at least 94% of the linkages are β-glycosidic bonds. In some embodiments, at least 95% of the linkages are β-glycosidic bonds. In some embodiments, at least 96% of the linkages are β-glycosidic bonds. In some embodiments, at least 97% of the linkages are β-glycosidic bonds. In some embodiments, at least 98% of the linkages are β-glycosidic bonds. In some embodiments, at least 99% of the linkages are β-glycosidic bonds. The diluent can be water soluble.

In some embodiments, 75% to 99% of the linkages are β-glycosidic bonds. In some embodiments, 85% to 99% of the linkages are β-glycosidic bonds. In some embodiments, 90% to 99% of the linkages are β-glycosidic bonds.

The β-glycosidic bond is selected from the group consisting of a β-1,1 linkage, a β-1,2 linkage, a β-1,3 linkage, a β-1,4 linkage, a β-1,5 linkage, and a β-1,6 linkage. The diluent can include the same type of β-glycosidic bond or two or more different types of β-glycosidic bond. In some embodiments, the diluent is substantially free of α-glycosidic bonds, e.g., less than 5% of α-glycosidic bonds, less than 2% of α-glycosidic bonds, less than 1% of α-glycosidic bonds, less than 0.5% of α-glycosidic bonds, less than 0.4% of α-glycosidic bonds, less than 0.3% of α-glycosidic bonds, less than 0.2% of α-glycosidic bonds, or less than 0.1% of α-glycosidic bonds.

Exemplary diluents where at least at least 70% of the linkages between the monosaccharide units are β-glycosidic bonds include, but are not limited to, carboxymethylcellulose, chitosan, chrysoloaminarin, curdlan, laminarin, lentinan, lichenin, pleuran, zymosan, oat beta glucan, wheat beta glucan, rye beta glucan, barley beta glucan, chondroitin sulfate, or a combination thereof. In some embodiments, the diluent is carboxymethylcellulose. In some embodiments, the diluent is oat beta glucan. In some embodiments, the diluent is lentinan. In some embodiments, the diluent is a combination of carboxymethylcellulose and oat beta glucan. The weight ratio of carboxymethylcellulose and oat beta glucan can be 1:99 to 99:1. In some embodiments, the diluent is a combination of carboxymethylcellulose and lentinan. The weight ratio of carboxymethylcellulose and lentinan can be 1:99 to 99:1. In some embodiments, the diluent is a combination of oat beta glucan and lentinan. The weight ratio of oat beta glucan and lentinan can be 1:99 to 99:1. In some embodiments, the diluent is a combination of carboxymethylcellulose, oat beta glucan, and lentinan.

In another aspect, the present disclosure provides another dry composition that includes: (a) a bacterial composition including one or more lactic-acid-producing bacteria having a bacterial concentration of at least 1×10⁶ CFU per gram of the bacterial composition; and (b) a diluent, wherein the diluent includes a plurality of monosaccharide units connected by a plurality of linkages, each monosaccharide unit being connected to an adjacent monosaccharide unit by one of the linkages, where at least 70% of the linkages are α-glycosidic bonds that are resistant to hydrolysis by an amylase. Such diluent is indigestible when consumed. The dry composition is stable, e.g., at room temperature. For example, at least 80% of the bacterial activity is retained over a period of at least 15 days, e.g., at least 20 days, at least 25 days, at least 30 days, at least 35 days, at least 40 days, at least 45 days, at least 50 days, at least 55 days, or at least 60 days. In some embodiments, at least 90% of the bacterial activity is retained over a period of at least 15 days, e.g., at least 20 days, at least 25 days, at least 30 days, at least 35 days, at least 40 days, at least 45 days, at least 50 days, at least 55 days, or at least 60 days. In some embodiments, at least 95% of the bacterial activity is retained over a period of at least 15 days, e.g., at least 20 days, at least 25 days, at least 30 days, at least 35 days, at least 40 days, at least 45 days, at least 50 days, at least 55 days, or at least 60 days.

In some embodiments, at least 75% of the linkages are α-glycosidic bonds that are resistant to hydrolysis by an amylase. In some embodiments, at least 80% of the linkages are α-glycosidic bonds that are resistant to hydrolysis by an amylase. In some embodiments, at least 85% of the linkages are α-glycosidic bonds that are resistant to hydrolysis by an amylase. In some embodiments, at least 90% of the linkages are α-glycosidic bonds that are resistant to hydrolysis by an amylase. In some embodiments, at least 91% of the linkages are α-glycosidic bonds that are resistant to hydrolysis by an amylase. In some embodiments, at least 92% of the linkages are α-glycosidic bonds that are resistant to hydrolysis by an amylase. In some embodiments, at least 93% of the linkages are α-glycosidic bonds that are resistant to hydrolysis by an amylase. In some embodiments, at least 94% of the linkages are α-glycosidic bonds that are resistant to hydrolysis by an amylase. In some embodiments, at least 95% of the linkages are α-glycosidic bonds that are resistant to hydrolysis by an amylase. In some embodiments, at least 96% of the linkages are α-glycosidic bonds that are resistant to hydrolysis by an amylase. In some embodiments, at least 97% of the linkages are α-glycosidic bonds that are resistant to hydrolysis by an amylase. In some embodiments, at least 98% of the linkages are α-glycosidic bonds that are resistant to hydrolysis by an amylase. In some embodiments, at least 99% of the linkages are α-glycosidic bonds that are resistant to hydrolysis by an amylase. The diluent can be water-soluble.

In some embodiments, 75% to 99% of the linkages are α-glycosidic bonds that are resistant to hydrolysis by an amylase. In some embodiments, 85% to 99% of the linkages are α-glycosidic bonds that are resistant to hydrolysis by an amylase. In some embodiments, 90% to 99% of the linkages are α-glycosidic bonds that are resistant to hydrolysis by an amylase.

The α-glycosidic bond is selected from the group consisting of an α-1,1 linkage, an α-1,2 linkage, an α-1,3 linkage, an α-1,4 linkage, an α-1,5 linkage, and an α-1,6 linkage. The diluent can include the same type of α-glycosidic bond or two or more different types of α-glycosidic bond.

Exemplary diluents where at least 70% of the linkages between the monosaccharide units are α-glycosidic bonds that are resistant to hydrolysis by an amylase include, but are not limited to, digestion-resistant maltodextrin, digestion-resistant inulin, digestion-resistant starch, or a combination thereof. In some embodiments, the diluent is Fibersol®-2. In some embodiments, the diluent is Nutriose®. In some embodiments, the diluent is a combination of Fibersol®-2 and Nutriose®. The weight ratio of Fibersol®-2 and Nutriose® can be 1:99 to 99:1.

In some embodiments of the dry compositions disclosed herein, the diluent can have a molecular weight at least 10,000 Dalton, e.g., at least 11,000 Dalton, at least 12,000 Dalton, at least 13,000 Dalton, at least 14,000 Dalton, at least 15,000 Dalton, at least 20,000 Dalton, at least 25,000 Dalton, or at least 30,000 Dalton.

In some embodiments of the dry compositions disclosed herein, the diluent can have a molecular weight no more than 500,000 Dalton, e.g., no more than 400,000 Dalton, no more than 300,000 Dalton, no more than 200,000 Dalton, no more than 100,000 Dalton, no more than 90,000 Dalton, no more than 80,000 Dalton, no more than 70,000 Dalton, no more than 60,000 Dalton, or no more than 50,000 Dalton.

In some embodiments of the dry compositions disclosed herein, the diluent can have a molecular weight in the range of 10,000 to 500,000 Dalton, e.g., 10,000 to 400,000 Dalton, 10,000 to 300,000 Dalton, 10,000 to 200,000 Dalton, 10,000 to 100,000 Dalton, 10,000 to 90,000 Dalton, 10,000 to 80,000 Dalton, 10,000 to 70,000 Dalton, 10,000 to 60,000 Dalton, 10,000 to 50,000 Dalton, 10,000 to 40,000 Dalton, 10,000 to 30,000 Dalton, or 10,000 to 25,000 Dalton.

In some embodiments of the dry compositions disclosed herein, the diluent can include 40 to 3000 monosaccharide units, e.g., 40 to 2000, 40 to 1500, 40 to 1000, 40 to 750, 40 to 500, 40 to 450, 40 to 400, 40 to 350, 40 to 300, 40 to 250, 40 to 200, 100 to 300, 100 to 250, or 100 to 200 monosaccharide units.

In some embodiments of the dry compositions disclosed herein, the diluent can have a general formula of Cₓ(H₂O)_{y}, where x is between 200 and 2500, and y is between 200 and 2500. In some embodiments, x is between 200 and 2000, between 200 and 1500, between 200 and 1000, or between 500 and 2500. In some embodiments, y is between 200 and 2000, between 200 and 1500, between 200 and 1000, or between 500 and 2500.

In some embodiments of the dry compositions disclosed herein, the diluent can have a general formula of (C₆H₁₀O₅)ₙ, where n is between 40 and 3000. In some embodiments, n is between 40 and 2000, between 40 and 1500, between 40 and 1000, between 40 and 750, between 40 and 500, between 40 and 450, between 40 and 400, between 40 and 350, between 40 and 300, between 40 and 250, between 40 and 200, between 100 and 500, between 100 and 400, between 100 and 300, between 100 and 250, or between 100 and 200.

In some embodiments, the monosaccharide units can be connected in a linear manner. In some embodiments, the diluent can have a linear backbone with one or more branches.

In some embodiments of the dry compositions disclosed herein, the one or more lactic-acid-producing bacteria is Lactobacillus Plantarum, Pediococcus acidilactici, Pediococcus pentosaceus, or a mixture thereof. In some embodiments, the dry composition includes a bacterial composition including Lactobacillus Plantarum, Pediococcus acidilactici, and Pediococcus pentosaceus. In some embodiments, the Pediococcus acidilactici, Pedococcus pentosaceus, and Lactobacillus plantarum are present in equal proportions by CFU count.

The bacterial composition of the dry compositions described herein can further include one or more Bacillus strains. Exemplary Bacillus strains include, but are not limited to, Bacillus subtilis, Bacillus licheniformis, Bacillus pumilus, Bacillus amyloliquefaciens, Bacillus Mojavensis, Bacillus coagulans, and Bacillus clausii. For example, the bacterial mixture can further include Bacillus subtilis 34 KLB.

Bacillus subtilis strain 34KLB
(SEQ ID NO.: 1)
AGCTCGGATCCACTAGTAACGGCCGCCAGTGTGCTGGAATTCGCCCTTAG
AAAGGAGGTGATCCAGCCGCACCTTCCGATACGGCTACCTTGTTACGACT
TCACCCCAATCATCTGTCCCACCTTCGGCGGCTGGCTCCATAAAGGTTAC
CTCACCGACTTCGGGTGTTACAAACTCTCGTGGTGTGACGGGCGGTGTGT
ACAAGGCCCGGGAACGTATTCACCGCGGCATGCTGATCCGCGATTACTAG
CGATTCCAGCTTCACGCAGTCGAGTTGCAGACTGCGATCCGAACTGAGAA
CAGATTTGTGRGATTGGCTTAACCTCGCGGTTTCGCTGCCCTTTGTTCTG
TCCATTGTAGCACGTGTGTAGCCCAGGTCATAAGGGGCATGATGATTTGA
CGTCATCCCCACCTTCCTCCGGTTTGTCACCGGCAGTCACCTTAGAGTGC
CCAACTGAATGCTGGCAACTAAGATCAAGGGTTGCGCTCGTTGCGGGACT
TAACCCAACATCTCACGACACGAGCTGACGACAACCATGCACCACCTGTC
ACTCTGCCCCCGAAGGGGACGTCCTATCTCTAGGATTGTCAGAGGATGTC
AAGACCTGGTAAGGTTCTTCGCGTTGCTTCGAATTAAACCACATGCTCCA
CCGCTTGTGCGGGCCCCCGTCAATTCCTTTGAGTTTCAGTCTTGCGACCG
TACTCCCCAGGCGGAGTGCTTAATGCGTTAGCTGCAGCACTAAAGGGGCG
GAAACCCCCTAACACTTAGCACTCATCGTTTACGGCGTGGACTACCAGGG
TATCTAATCCTGTTCGCTCCCCACGCTTTCGCTCCTCAGCGTCAGTTACA
GACCAGAGAGTCGCCTTCGCCACTGGTGTTCCTCCACATCTCTACGCATT
TCACCGCTACACGTGGAATTCCACTCTCCTCTTCTGCACTCAAGTTCCCC
AGTTTCCAATGACCCTCCCCGGTTGAGCCGGGGGCTTTCACATCAGACTT
AAGAAACCGCCTGCGAGCCCTTTACGCCCAATAAtTCCGGACAACGCTTG
CCACCTACGTATTACCGCGGCTGCTGGCACGTAGTTAGCCGTGGCTTTCT
GGTTAGGTACCGTCAAGGTGCCGCCCTATTTGAACGGCACTTGTTCTTCC
CTAACAACAGAGCTTTACGATCCGAAAACCTTCATCACTCACGCGGCGTT
GCTCCGTCAGACTTTCGTCCATTGCGGAAGATTCCCTACTGCTGCCTCCC
GTAGGAGTCTGGGCCGTGTCTCAGTCCCAGTGTGGCCGATCACCCTCTCA
GGTCGGCTACGCATCGTCGCCTTGGTGAGCCGTTACCTCACCAACTAGCT
AATGCGCCGCGGGTCCATCTGTAAGTGGTAGCCGAAGCCACCTTTTATGT
CTGAACCATGCGGTTCAGACAACCATCCGGTATTAGCCCCGGTTTCCCGG
AGTTATCCCAGTCTTACAGGCAGGTTACCCACGTGTTACTCACCCGTCCG
CCGCTAACATCAGGGAGCAAGCTCCCATCTGTCCGCTCGACTTGCATGTA
TTAGGCACGCCGCCAGCGTTCGTCCTGAGCCATGAACAAACTCTAAGGGC
GAATTCTGCAGATATCCATCACACTGGCGGCCGCTCGAGCATGCATCTAG
AGGGCCCAATCGCCCTAT

The bacterial mixture contains bacteria at a concentration between about 10⁶ and 10¹¹ CFUs per gram. In some embodiments, the bacteria are at a concentration of at least 10⁹ CFUs per gram. In some embodiments, the bacteria are at a concentration of at least 10¹⁰ CFUs per gram. In some embodiments, the bacteria are at a concentration of about 10⁹ to 10¹¹ CFUs per gram. Lactic acid counts can be obtained on MRS agar. Bacillus counts can be obtained on Trypticase soy agar.

The probiotic lactic-acid-producing bacteria according to the invention may be produced using any standard fermentation process known in the art. For example, solid substrate or submerged liquid fermentation. The fermented cultures can be mixed cultures, microbiotic composites or single isolates. The probiotic lactic-acid-producing bacteria are anaerobically fermented. The probiotic lactic-acid-producing bacteria can be a combination of solid substrate and a submerged liquid fermentation. In some embodiments, the probiotic lactic-acid-producing bacteria are anaerobically fermented in the presence of carbohydrates. Suitable carbohydrates include inulin, fructooligosaccharide, and glucooligosaccharides.

After fermentation, the bacteria can be harvested by any known methods in the art. For example, the bacteria can be harvested by filtration or centrifugation, or simply supplied as the ferment. The bacteria can be dried by any method known in the art. For example, the bacteria are dried by liquid nitrogen followed by lyophilization. The compositions according to the present disclosure are freeze dried to moisture content less than 20%, 15%, 10%, 9%, 8%, 7%, 6%, 5%, 4%, 3%, 2%, or 1% by weight. Preferably, the composition according to the invention has been freeze dried to moisture content less than 5% by weight. In some embodiments, the freeze-dried powder is ground to decrease the particle size. The bacteria are ground by conical grinding at a temperature less than 10° C., 9° C., 8° C., 7° C., 6° C., 5° C., 4° C., 3° C., 2° C., 1° C., 0° C., or less. Preferably, the temperature is less than 4° C.

In some embodiments, the probiotic bacteria are fermented anaerobically, dried, and ground into particles. The particles can have an average size of about 400 microns, about 300 microns, or about 200 microns. In some embodiments, the particles have a mean particle size of about 200 microns with 60% of the product in a size range between 175-840 microns. The particle size can be measured using sieving according to ANSI/ASAE 5319.4 method.

The ratio of the diluent to the lactic-acid-producing bacteria is at least 90:1 by weight, at least 91:1 by weight, at least 92:1 by weight, at least 93:1 by weight, at least 94:1 by weight, at least 95:1 by weight, at least 96:1 by weight, at least 97:1 by weight, at least 98:1 by weight, or at least 99:1 by weight.

The dry composition can include about 0.1% to 10% of the lactic-acid-producing bacteria by weight, e.g., about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, or 10% by weight. The dry composition can include about 0.1% to 5% of Bacillus subtilis 34 KLB by weight, e.g., about 0.1%, 0.2%, 0.3%, 0.4%, 0.5%, 0.6%, 0.7%, 0.8%, 0.9%, 1%, 2%, 3%, 4%, or 5% by weight. The dry composition can include about 90% to 99.9% of the diluent by weight, e.g., about 90%, 91%, 92%, 93%, 94%, 95%, 96%, 97%, 98%, or 99% by weight.

In some embodiments, the dry composition includes 0.4% of the lactic-acid-producing bacteria by weight, 0.15% of Bacillus subtilis 34 KLB by weight, and 99.45% of the diluent by weight.

The dry composition can further include a prebiotic selected from the group consisting of inulin, a fructooliogsaccharide, and a glucooligosaccharide. The dry composition can further include a postbiotic. The postbiotic can be prepared by drying the supernatant from the centrifugation of the anaerobic fermentation of a bacterial species. In some embodiments, the postbiotic is prepared, for example, by collecting the supernatants from the centrifugation of each of the individual anaerobic fermentations of Pediococcus acidilactici, Pediococcus pentosaceus, Lactobacillus plantarum; mixing the supernatants together; freeze-drying the mixed supernatants to a moisture content less than about 5% by weight. The resulting dried product can be ground to a mean particle size of 295 microns, with 60% of the mixture in the size range between 175-900 microns.

Dextrose, sucrose, mannose, soluble starch, and/or pectin can have an adverse effect on the stability of lactic acid bacteria. Accordingly, some embodiments of the dry compositions are substantially free of dextrose, sucrose, mannose, soluble starch, and/or pectin.

In some embodiments, the dry composition can include 5% or less dextrose by weight, 4% or less dextrose by weight, 3% or less dextrose by weight, 2% or less dextrose by weight, 1% or less dextrose by weight, 0.5% or less dextrose by weight, or 0.1% or less dextrose by weight.

In some embodiments, the dry composition can include 5% or less sucrose by weight, 4% or less sucrose by weight, 3% or less sucrose by weight, 2% or less sucrose by weight, 1% or less sucrose by weight, 0.5% or less sucrose by weight, or 0.1% or less sucrose by weight.

In some embodiments, the dry composition can include 5% or less mannose by weight, 4% or less mannose by weight, 3% or less mannose by weight, 2% or less mannose by weight, 1% or less mannose by weight, 0.5% or less mannose by weight, or 0.1% or less mannose by weight.

In some embodiments, the dry composition can include 5% or less soluble starch by weight, 4% or less soluble starch by weight, 3% or less soluble starch by weight, 2% or less soluble starch by weight, 1% or less soluble starch by weight, 0.5% or less soluble starch by weight, or 0.1% or less soluble starch by weight.

In some embodiments, the dry composition can include 5% or less pectin by weight, 4% or less pectin by weight, 3% or less pectin by weight, 2% or less pectin by weight, 1% or less pectin by weight, 0.5% or less pectin by weight, or 0.1% or less pectin by weight.

The dry compositions of the present disclosure can be formulated into different forms including chewable foods, tablets, capsules, powders, and granulates, e.g., for oral administration. In some embodiments, the dry compositions are formulated into a tablet. In some embodiments, the dry compositions are formulated into a capsule. In some embodiments, the dry compositions are formulated into granulated or water soluble powders.

When formulated, the dry composition may contain further ingredients, including ingredients that have a favorable impact on health, flavor, formulating or tableting. Non-limiting examples of additional ingredients that may suitably be incorporated in the present composition are: vitamins, minerals, nutritional supplements (e.g., fiber), fungal extracts, botanical extracts, sweeteners, flow aids, and fillers.

In some embodiments, the dry compositions of the present disclosure can further include minerals, vitamins, sugars, tableting aids, and flavouring.

Minerals include, for example, calcium carbonate, calcium lactate, calcium chloride, calcium phosphate (dibasic), sodium chloride, potassium citrate monohydrate, potassium sulfate, potassium phosphate monobasic, magnesium oxide, manganese carbonate, manganese glucanate, ferric citrate, zinc carbonate, zinc glucanate, cupric carbonate, potassium iodate, sodium selenite pentahydrate, chromium potassium sulfate dodecahydrate, ammonium paramolybdate tetrahydrate, sodium meta-silicate nonahydrate, lithium chloride, boric acid, sodium fluoride, nickel carbonate hydroxide tetrahydrate, or ammonium meta-vanadate. Minerals can be formulated at a concentration of about 0.1% to 10% w/w or 0.1% to 5.0% w/w or any specific value within said range. In some embodiments, minerals are formulated at a concentration of about 5% w/w, 4% w/w, 3% w/w, 2% w/w, 1% w/w, 0.9% w/w, 0.8% w/w, 0.7% w/w, 0.6% w/w, 0.5% w/w, 0.4% w/w, 0.3% w/w, 0.2% w/w, 0.1% w/w, or less.

Vitamins include for example, vitamin B1, B2, B3, B5, B6, B7, B9, B12, vitamin C, vitamin D, vitamin E, vitamin A and/or K. Preferred are vitamin B1, B3, B6, B12, vitamin C, vitamin D3, or vitamin E acetate. Vitamins are formulated at a concentration of about 0.01% to 10% w/w, 0.1% to 5% w/w, or any specific value within said range. In particular, vitamins are formulated at a concentration of about 5% w/w, 4% w/w, 3% w/w, 2% w/w, 1% w/w, 0.9% w/w, 0.8% w/w, 0.7% w/w, 0.6% w/w, 0.5% w/w, 0.4% w/w, 0.3% w/w, 0.2% w/w, 0.1% w/w, or less.

The sugars can be present in the dry composition at a concentration that does not destabilize the bacteria. For example, the sugars are formulated at a concentration of less than 5% w/w, e.g., less than 4% w/w, less than 3% w/w, less than 2% w/w, less than 1% w/w, less than 0.9% w/w, less than 0.8% w/w, less than 0.7% w/w, less than 0.6% w/w, or less than 0.5% w/w,

Tableting aids include for example, carboxylic acids such as malic, maleic, citric, iso-citric and succinic acids, and salts thereof, SiO₂, Aloe Vera, saturated and unsaturated linear and branched fatty acids and their salts, or fatty alcohols. Preferred tableting aides are malic acid, citric acid, stearic acid or magnesium stearate. Tableting aides are formulated at a concentration of about 1% to 10% w/w, 2.5% to 7.5% w/w, or any specific value within said range. In particular, tableting aides are formulated at a concentration of about 10% w/w, 7.5% w/w, 5% w/w, 4% w/w, 3% w/w, 2% w/w, 1% w/w, or less.

Any natural or artificial food grade flavorings may be used including banana, cinnamon, grape, orange, citrus, peach, pear, pineapple, apple, berry, coconut, chocolate, vanilla, strawberry, wintergreen, spearmint, peppermint, or ginger. In some embodiments, the flavorings are ginger or natural berry flavorings. Flavorings can be formulated at a concentration of about 0.1% to 10% w/w, 0.5% to 5% w/w, or any specific value within said range. In particular, flavoring agents are formulated at a concentration of about 5% w/w, 4% w/w, 3% w/w, 2% w/w, 1% w/w, 0.9% w/w, 0.8% w/w, 0.7% w/w, 0.6% w/w, 0.5% w/w, 0.4% w/w, 0.3% w/w, 0.2% w/w, 0.1% w/w or less.

Fungal extracts may include whole components or specific extracts from gandoderma lucidium, lentinus edodes, hericium erinaceous, agaricus blazei, cordyceps sinensis, coriolus veriscolor, coprinus comatus, or grifola frondosa.

Botanicals may include Rhodiola Rosea extract, Aloe Barbedensis, Yucca Schidigera, Aloe vera, Boswellia extract, Acacia Catechu, Scutellaria baicalensis, moringa, or turmeric.

The dry compositions of the present disclosure are useful in improving a subject's health or nutrition. In some embodiments, the subject can be healthy and will be taking the dry compositions of the present disclosure as diet supplements. In some embodiments, the subject has an illness and the dry compositions of the present disclosure can be consumed to alleviate one or more symptoms associated with the illness.

The dry compositions of the present disclosure are also useful in methods of treating various disorders in a subject in need thereof. Specifically, the dry compositions can be used to promote digestive health, metabolism (nutritional heath), and weight management when administered orally. For example, the dry composition can be used to treat or alleviate a sign or symptom of a digestive disorder such as constipation (e.g. irritable bowel syndrome with constipation (IBS-C) or chronic idiopathic constipation (CIC)), diarrhea (e.g. chronic diarrhea), dysbiosis, Crohn's disease, food allergy, lactose intolerance, and chronic gastrointestinal inflammatory diseases such as pouchitis and ulcerative colitis. The dry compositions can also be used to reduce morbidities associated with metabolic syndrome X. The dry compositions can be administered orally as a tablet, capsule, powder, or granulate. Each tablet, capsule, powder, or granulate can contain between about 100-800 milligrams, e.g., about 100 mg, 150 mg, 200 mg, 250 mg, 300 mg, 350 mg, 400 mg, 450 mg, 500 mg, 550 mg, 600 mg, 650 mg, 700 mg, 750 mg, or 800 mg.

The dry compositions of the present disclosure can also be used as animal feeds. The dry compositions of the present disclosure can be formulated for administration in animal feed, including mash and pellets, but may also be delivered via the drinking water supply. In some embodiments, the dry compositions can be formulated into feed mash. In some embodiments, the dry compositions can be formulated into a feed pellet. In some embodiments, the dry compositions can be directly added to an animal's drinking water supply. These dry compositions improve animal health, including accelerating weight gain, reducing the waste odor and/or increasing utilization of feed proteins. For example, the dry compositions can improve animal health by increasing the weight of the animal by at least 10%, at least 20%, at least 30%, at least 40%, or at least 50% as compared to the same type of animals without being fed with the dry compositions.

When formulated for animal feed, the dry compositions are typically dosed between 0.1 and 1 kg per metric ton of feed. For example, the feed composition is dosed at 50, 100, 200, 300, 400, 500, 600, 700, 800, 900 or more grams per metric ton of feed. When dosed into drinking water, the dry compositions are typically dosed between 0.1 and 1 kg per 1000 liters. For aquatic applications, e.g., shrimp or fin fish, the dry compositions are typically dosed between 0.1 and 10 ppm, e.g., 0.1, 0.2, 0.25, 0.5, 1, 1.5, 2, 2.5, 3, 3.5, 4, 4.5, 5, or 5.5 ppm.

In some embodiments, the dry composition of the present disclosure is added on top of a diet at about 0.05% to 5% w/w, e.g., about 0.05%, 0.1%, 0.2%, 0.3%, 0.4%, 0.5%, 0.6%, 0.7%, 0.8%, 0.9%, 1.0%, 2.0%, 3.0%, 4.0%, or 5.0% w/w.

In another aspect, the present disclosure provides methods for stabilizing bacterial probiotic compositions such as lactic-acid-producing bacteria. In some embodiments, the method including contacting the lactic-acid-producing bacteria with a diluent having a plurality of monosaccharide units connected by a plurality of linkages, each monosaccharide unit being connected to an adjacent monosaccharide unit by one of the linkages, wherein at least 70% of the linkages are β-glycosidic bonds.

The present disclosure also provides a method of stabilizing lactic-acid-producing bacteria, the method includes contacting the lactic-acid-producing bacteria with a diluent having a plurality of monosaccharide units connected by a plurality of linkages, each monosaccharide unit being connected to an adjacent monosaccharide unit by one of the linkages, wherein at least 70% of the linkages are α-glycosidic bonds that are resistant to hydrolysis by an amylase.

In some embodiments of any one of the methods described herein, the lactic-acid-producing bacteria are mixed with the diluent to produce a mixture. The ratio of the diluent to the lactic-acid-producing bacteria can be at least 90:1 by weight, at least 95:1 by weight, at least 98:1 by weight, or at least 99:1 by weight.

In some embodiments of any one of the methods described herein, the lactic-acid-producing bacteria is Lactobacillus Plantarum, Pediococcus acidilactici, Pediococcus pentosaceus, or a mixture thereof. In some embodiments, the lactic-acid-producing bacteria can be in the form of a powder.

The methods described herein can stabilize lactic-acid-producing bacteria at about 5° C. to 40° C., e.g., 10° C. to 40° C. or 20° C. to 40° C. In some embodiments, the methods can stabilize lactic-acid-producing bacteria at room temperature.

The methods described herein can stabilize lactic-acid-producing bacteria over an extended period of time, e.g., 7 days to 365 days, 7 days to 240 days, 7 days to 120 days, 7 days to 90 days, or 30 days to 90 days. In some embodiments, the methods described herein can retain the bacterial activity of the lactic-acid-producing bacteria by at least 70% for at least 30 days. In some embodiments, the methods described herein can retain the bacterial activity of the lactic-acid-producing bacteria by at least 80% for at least 30 days. In some embodiments, the methods described herein can retain the bacterial activity of the lactic-acid-producing bacteria by at least 85% for at least 30 days. In some embodiments, the methods described herein can retain the bacterial activity of the lactic-acid-producing bacteria by at least 90% for at least 30 days. In some embodiments, the methods described herein can retain the bacterial activity of the lactic-acid-producing bacteria by at least 95% for at least 30 days. In some embodiments, the methods described herein can retain the bacterial activity of the lactic-acid-producing bacteria by at least 97% for at least 30 days.

The details of the invention are set forth in the accompanying description below. Although methods and materials similar or equivalent to those described herein can be used in the practice or testing of the present invention, illustrative methods and materials are now described. Other features, objects, and advantages of the invention will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this invention belongs. All patents and publications cited in this specification are incorporated herein by reference in their entireties.

### Definitions

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The articles “a” and “an” are used in this disclosure to refer to one or more than one (i.e., to at least one) of the grammatical object of the article. By way of example, “an element” means one element or more than one element.

As used herein, the term “and/or” is used in this disclosure to mean either “and” or “or” unless indicated otherwise.

As used herein, the term “prebiotic” as used herein includes compounds that stimulate the growth and or activity of bacteria.

By the term “postbiotic” as used herein refer to the non-viable bacterial products or metabolic byproducts from the probiotic organism.

As used herein, the term “about” refer to a range of values that are similar to the stated reference value. In certain embodiments, the term “about” refers to a range of values that fall within 10 percent or less (e.g., 10%, 9%, 8%, 7%, 6%, 5%, 4%, 3%, 2%, or 1%) of the stated reference value.

As used herein, the term “resistant to hydrolysis by an amylase,” when used to describe a bond or molecule, refers to the ability of the bond or molecule to avoid chemical breakdown due to contact with water in the presence of an amylase under normal physiological conditions. The amylase can be α-amylase, β-amylase, or γ-amylase.

As used herein, the term “subject” refers to a mammal or a non-mammal. The mammal can be a human, non-human primate, mouse, rat, dog, cat, horse, or cow, but are not limited to these examples. The non-mammal can be fish (e.g., crayfish, bottom dwelling fish, or finfish), shrimp, lobster, prawns, oysters, mussels, cockles, and mollusks. A subject can be male or female.

### EXAMPLES

The disclosure is further illustrated by the following examples and synthesis examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

### Example 1. Preparation of the Microbial Species

The microbial species of the present invention may be made by any of the standard fermentation processes known in the art. In the following examples, submerged liquid fermentation processes are described, however, where appropriate, solid substrate processes may be used.

Individual, purified isolates of Pediococcus acidilactici, Pediococcus pentosaceus, and Lactobacillus plantarum were grown-up in separate fermenters using standard anaerobic, submerged liquid fermentation protocols. After fermentation, the individual cultures were filtered, centrifuged, freeze dried to a moisture level less than about 5%, then ground to a mean particle size of about 200 microns with 60% of the product in a size range between 175-840 microns. The individual dried microbial cultures were then blended together at equal CFU count to obtain a microbial composition in accordance with some embodiments of the present disclosure. The final microbial concentration of the mixed powdered product is between 10⁹ and 10¹¹ CFU/g.

### Example 2. Microbial Formulations

The compositions shown in Table 1 were prepared by blending the dry ingredients in a rotary mixer. The final bacterial count of each composition was nominally 3.1×10⁸ CFU/g.

TABLE 1

| | COMPOSITIONS | | | | | | | | | |
|---|---|---|---|---|---|---|---|---|---|---|
| Ingredients | A | B | C | D | E | F | G | H | I | J |
| Dried lactic-acid- | 0.40 | 0.40 | 0.40 | 0.40 | 0.40 | 0.40 | 0.40 | 0.40 | 0.40 | 0.40 |
| producing | | | | | | | | | | |
| bacteria Mix from | | | | | | | | | | |
| Example 1 | | | | | | | | | | |
| Bacillus subtilis | 0.15 | 0.15 | 0.15 | 0.15 | 0.15 | 0.15 | 0.15 | 0.15 | 0.15 | 0.15 |
| 34 KLB | | | | | | | | | | |
| Dextrose | 99.45 | | | | | | | | | |
| Sucrose | | 99.45 | | | | | | | | |
| Mannose | | | 99.45 | | | | | | | |
| Soluble Starch | | | | 99.45 | | | | | | |
| Pectin | | | | | 99.45 | | | | | |
| Sodium | | | | | | 99.45 | | | | |
| Carboxymethylcellulose | | | | | | | | | | |
| (Sigma | | | | | | | | | | |
| Aldrich Mw | | | | | | | | | | |
| ~90,000) | | | | | | | | | | |
| Chitosan | | | | | | | 99.45 | | | |
| Oat Beta Glucan | | | | | | | | 99.45 | | |
| Chondroitin | | | | | | | | | 99.45 | |
| Sulfate | | | | | | | | | | |
| Xanthan gum | | | | | | | | | | 99.45 |
| Lentinan | | | | | | | | | | |
| Gelatin | | | | | | | | | | |
| Alginate | | | | | | | | | | |
| Polyethylene | | | | | | | | | | |
| Glycol (800) | | | | | | | | | | |
| Polyvinyl alcohol | | | | | | | | | | |
| (Polysciences, | | | | | | | | | | |
| Inc., Mw | | | | | | | | | | |
| ~31,000, 88% | | | | | | | | | | |
| hydrolyzed) | | | | | | | | | | |
| Montmorillonite | | | | | | | | | | |
| clay | | | | | | | | | | |
| Fibersol ®-2 | | | | | | | | | | |
| Inulin LV 110 | | | | | | | | | | |
| Nutriose ® | | | | | | | | | | |

| | COMPOSITIONS | | | | | | | | |
|---|---|---|---|---|---|---|---|---|---|
| Ingredients | K | L | M | N | O | P | Q | R | S |
| Dried lactic-acid- | 0.40 | 0.40 | 0.40 | 0.40 | 0.40 | 0.40 | 0.40 | 0.40 | 0.40 |
| producing | | | | | | | | | |
| bacteria Mix from | | | | | | | | | |
| Example 1 | | | | | | | | | |
| Bacillus subtilis | 0.15 | 0.15 | 0.15 | 0.15 | 0.15 | 0.15 | 0.15 | 0.15 | 0.15 |
| 34 KLB | | | | | | | | | |
| Dextrose | | | | | | | | | |
| Sucrose | | | | | | | | | |
| Mannose | | | | | | | | | |
| Soluble Starch | | | | | | | | | |
| Pectin | | | | | | | | | |
| Sodium | | | | | | | | | |
| Carboxymethylcellulose | | | | | | | | | |
| (Sigma | | | | | | | | | |
| Aldrich Mw | | | | | | | | | |
| ~90,000) | | | | | | | | | |
| Chitosan | | | | | | | | | |
| Oat Beta Glucan | | | | | | | | | |
| Chondroitin | | | | | | | | | |
| Sulfate | | | | | | | | | |
| Xanthan gum | | | | | | | | | |
| Lentinan | 99.45 | | | | | | | | |
| Gelatin | | 99.45 | | | | | | | |
| Alginate | | | 99.45 | | | | | | |
| Polyethylene | | | | 99.45 | | | | | |
| Glycol (800) | | | | | | | | | |
| Polyvinyl alcohol | | | | | 99.45 | | | | |
| (Polysciences, | | | | | | | | | |
| Inc., Mw | | | | | | | | | |
| ~31,000, 88% | | | | | | | | | |
| hydrolyzed) | | | | | | | | | |
| Montmorillonite | | | | | | 99.45 | | | |
| clay | | | | | | | | | |
| Fibersol ®-2 | | | | | | | 99.60 | | |
| Inulin LV 110 | | | | | | | | 99.60 | |
| Nutriose ® | | | | | | | | | 99.60 |

### Example 3. Storage Stability Testing

Each of the compositions from Example 2 were packaged in foil-lined bags and placed in a 30° C. constant temperature room at ambient relative humidity. 10-gram samples were pulled at regular intervals and tested for residual lactic-acid-producing bacteria and bacillus activity using standard microbiology plating protocols. Lactic acid counts were obtained on EMD Millipore-supplied MRS-agar. Bacillus counts were obtained on Trypticase Soy Agar. The recovered lactic-acid-producing bacterial counts are shown in Table 2.

TABLE 2

| | Recovered lactic-acid-producing bacterial Counts (CFU/g) | | | |
|---|---|---|---|---|
| Composition | Day 0 | Day 30 | Day 64 | Day 96 |
| A | 3.00 × 10⁸ | 1.00 × 10² | n.d.* | n.d. |
| B | 2.43 × 10⁸ | 1.00 × 10² | n.d. | n.d. |
| C | 2.90 × 10⁸ | n.d. | n.d. | n.d. |
| D | 9.00 × 10⁶ | 3.40 × 10² | n.d. | n.d. |
| E | 3.00 × 10⁸ | n.d. | n.d. | n.d. |
| F | 5.00 × 10⁸ | 1.22 × 10⁸ | 2.30 × 10⁸ | 4.46 × 10⁷ |
| G | 2.70 × 10⁸ | 6.46 × 10⁶ | 1.76 × 10⁶ | 1.14 × 10⁶ |
| H | 2.97 × 10⁸ | 2.19 × 10⁸ | 4.37 × 10⁸ | 2.30 × 10⁸ |
| I | 2.37 × 10⁸ | 3.63 × 10⁷ | 3.27 × 10⁷ | 1.01 × 10⁷ |
| J | 9.03 × 10⁷ | 3.63 × 10⁴ | 5.80 × 10³ | |
| K | 7.03 × 10⁸ | 1.10 × 10⁸ | 5.60 × 10⁷ | 4.27 × 10⁷ |
| L | 2.43 × 10⁸ | 1.33 × 10³ | 3.62 × 10² | |
| M | 4.87 × 10⁷ | 3.60 × 10⁴ | 5.70 × 10³ | 1.70 × 10³ |
| N | 5.73 × 10⁸ | 1.10 × 10⁷ | 8.67 × 10⁵ | 6.57 × 10⁴ |
| O | 1.30 × 10⁸ | 1.68 × 10³ | 6.67 | n.d. |
| P | 6.63 × 10⁷ | n.d. | n.d. | n.d. |
| Q | 4.97 × 10⁸ | 2.17 × 10⁸ | | |
| R | 4.10 × 10⁸ | 5.50 × 10⁵ | | |
| S | 7.00 × 10⁸ | 1.67 × 10⁸ | | |

*n.d. = not detected

### EQUIVALENTS

While the present invention has been described in conjunction with the specific embodiments set forth above, many alternatives, modifications and other variations thereof will be apparent to those of ordinary skill in the art. All such alternatives, modifications and variations are intended to fall within the spirit and scope of the present invention.

1. A dry composition comprising:
a. 5% or less by weight of a bacterial composition comprising one or more lactic-acid-producing bacteria having a bacterial concentration of at least 1×10⁶ colony forming units (CFU) per gram of the bacterial composition; and
b. at least 95% by weight of a diluent, wherein the diluent comprises a plurality of monosaccharide units connected by a plurality of linkages, each monosaccharide unit being connected to an adjacent monosaccharide unit by one of the linkages, wherein at least 70% of the linkages are β-glycosidic bonds,
wherein at least 80% of the bacterial activity in the dry composition is retained over a period of 30 days.

2. The dry composition of claim 1, wherein the bacterial composition further comprises a Bacillus bacterial species.

3. The dry microbial composition of claim 2, wherein the Bacillus bacterial species is Bacillus subtilis 34 KLB.

4. The dry composition of claim 1, wherein at least 80% of the linkages between the monosaccharide units are β-glycosidic bonds.

5. The dry composition of claim 4, wherein at least 90% of the linkages between the monosaccharide units are β-glycosidic bonds.

6. The dry composition of claim 1, wherein the β-glycosidic bond is selected from the group consisting of a β-1,1 linkage, a β-1,2 linkage, a β-1,3 linkage, a β-1,4 linkage, a β-1,5 linkage, and a β-1,6 linkage.

7. The dry composition of claim 1, wherein the one or more lactic-acid-producing bacteria is Lactobacillus Plantarum, Pediococcus acidilactici, Pediococcus pentosaceus, or a mixture thereof.

8. The dry composition of claim 7, wherein each of the Pediococcus acidilactici, Pediococcus pentosaceus, and Lactobacillus plantarum is fermented anaerobically, dried, and ground to an average particle size of about 200 microns.

9. The dry composition of claim 7, wherein the Lactobacillus Plantarum, Pediococcus acidilactici, Pediococcus pentosaceus are present at equal CFU count per gram of the composition.

10. The dry composition of claim 1, wherein the diluent is carboxymethylcellulose, chitosan, chrysoloaminarin, curdlan, laminarin, lentinan, lichenin, pleuran, zymosan, oat beta glucan, wheat beta glucan, rye beta glucan, barley beta glucan, chondroitin sulfate, or a mixture thereof.

11. The dry composition of claim 1, further comprising a prebiotic selected from the group consisting of inulin, a fructooliogsaccharide, and a glucooligosaccharide.

12. The dry composition of claim 1, further comprising a vitamin, a mineral, a sugar, a botanical, or a fungal component.

13. The dry composition of claim 12, wherein the vitamin is vitamin A, vitamin B1, vitamin B2, vitamin B3, vitamin B5, vitamin B6, vitamin B7, vitamin B9, vitamin B12, vitamin C, vitamin D, vitamin E, or vitamin K.

14. The dry composition of claim 12, wherein the mineral is diatomaceous earth, calcium carbonate, calcium lactate, calcium chloride, calcium phosphate (dibasic), sodium chloride, potassium citrate monohydrate, potassium sulfate, potassium phosphate monobasic, magnesium oxide, manganese carbonate, manganese gluconate, ferric citrate, zinc carbonate, zinc gluconate, cupric carbonate, potassium iodate, sodium selenite pentahydrate, chromium potassium sulfate dodecahydrate, ammonium paramolybdate tetrahydrate, sodium meta-silicate nonahydrate, lithium chloride, boric acid, sodium fluoride, nickel carbonate hydroxide tetrahydrate, or ammonium meta-vanadate.

15. The dry composition of claim 12, wherein the botanical is rhodiola rosea extract, acadia catechu, scutellaria baicalensis, moringa, or turmeric.

16. The dry composition of claim 12, wherein the fungal component is ganoderma lucidium, lentinus edodes, hericium erinaceuous, agaricus blazei, cordyceps sinensis, coriolus versicolor, corprinus comatus, or grifola frondosa.

17. The dry composition of claim 1, having a moisture content of less than 5% by weight.

18. The dry composition of claim 1, wherein the bacterial activity in the dry composition is determined by the bacterial concentration.

19. A dry composition comprising:
a. 5% or less by weight of a bacterial composition comprising one or more lactic-acid-producing bacteria having a bacterial concentration of at least 1×10⁶ colony forming units (CFU) per gram of the bacterial composition; and
b. at least 95% by weight of a diluent, wherein the diluent comprises a plurality of monosaccharide units connected by a plurality of linkages, each monosaccharide unit being connected to an adjacent monosaccharide unit by one of the linkages, where at least 70% of the linkages are α-glycosidic bonds that are resistant to hydrolysis by an amylase,
wherein at least 80% of the bacterial activity in the dry composition is retained over a period of 30 days.

20-36. (canceled)

37. A method of stabilizing lactic-acid-producing bacteria, the method comprising contacting the lactic-acid-producing bacteria with a diluent having a plurality of monosaccharide units connected by a plurality of linkages, each monosaccharide unit being connected to an adjacent monosaccharide unit by one of the linkages, wherein at least 70% of the linkages are β-glycosidic bonds.

38. The method of claim 37, wherein at least 80% of the linkages between the monosaccharide units are β-glycosidic bonds.

39. The method of claim 37, wherein at least 90% of the linkages between the monosaccharide units are β-glycosidic bonds.

40. The method of claim 37, wherein the β-glycosidic bond is selected from the group consisting of a β-1,1 linkage, a β-1,2 linkage, a β-1,3 linkage, a β-1,4 linkage, a β-1,5 linkage, and a β-1,6 linkage.

41. The method of claim 37, wherein the lactic-acid-producing bacteria is Lactobacillus Plantarum, Pediococcus acidilactici, Pediococcus pentosaceus, or a mixture thereof.

42. The method of claim 37, wherein the diluent is carboxymethylcellulose, chitosan, chrysoloaminarin, curdlan, laminarin, lentinan, lichenin, pleuran, zymosan, oat beta glucan, wheat beta glucan, rye beta glucan, barley beta glucan, chondroitin sulfate, or a mixture thereof.

43. The method of claim 37, wherein the ratio of the diluent to the lactic-acid-producing bacteria is at least 95:1 by weight.

44. The method of claim 37, wherein the ratio of the diluent to the lactic-acid-producing bacteria is at least 99:1 by weight.

45. The method of claim 37, wherein at least 80% of the bacterial activity is retained.

46. The method of claim 45, wherein at least 90% of the bacterial activity is retained.

47. The method of claim 37, wherein the diluent is substantially free of α-glycosidic bonds.

48. A method of stabilizing lactic-acid-producing bacteria, the method comprising contacting the lactic-acid-producing bacteria with a diluent having a plurality of monosaccharide units connected by a plurality of linkages, each monosaccharide unit being connected to an adjacent monosaccharide unit by one of the linkages, wherein at least 70% of the linkages are α-glycosidic bonds that are resistant to hydrolysis by an amylase.

49-57. (canceled)

58. A method of improving a subject's health or nutrition, the method comprising administering to the subject the composition of claim 1 or 19.

59. The method of claim 58, wherein the composition is administered orally.

60. The method of claim 58, wherein the composition is administered through animal feed or drinking water when the subject is an animal.

61. The method of claim 58, wherein the composition is administered as a tablet, capsule, powder, or granulate.

62. The method of claim 61, wherein each tablet, capsule, powder, or granulate contains between 100-800 milligrams of the composition.

63. A method for treating a digestive disorder in a subject in need thereof, the method comprising administering the composition of claim 1 or 19 orally.

64. The method of claim 63, wherein the digestive disorder is constipation, diarrhea, dysbiosis, Crohn's disease, food allergy, lactose intolerance, pouchitis, or ulcerative colitis.
